(19) United States
(12) Patent Application Publication
(10) Pub. No.: US 2007/0082020 A1
(43) Pub. Date: Apr. 12, 2007

(54) Blood clot filter having coating

(76) Inventor: Vincent A. Piccone JR., Staten Island, NY (US)

Correspondence Address: Louis A. Piccone, 519 Kirchner Road, Dalton, MA, 01226, US

(21) Appl. No.: 11/248,024

(22) Filed: Oct. 11, 2005

(51) Int. Cl. A61F 2/02

(52) U.S. Cl. 424/423; 514/283; 514/449; 514/369; 514/410; 514/109; 514/49; 514/251; 514/269

(57) ABSTRACT

The present invention is directed to a blood clot filter for implantation in a patient comprising a blood clot filter having a coating comprising one or more pharmaceutically active compounds.

The present invention is directed to a medical device comprising a blood clot filter for insertion into a vein or artery of a patient wherein the blood clot filter has a coating.

For purposes of this invention a blood clot filter is a medical devise for insertion in a patient which prevents or minimizes a blood clots movement in the patients circulatory system.

The blood clot filter can be any blood clot filter serving the purpose of preventing, minimizing or restricting, the movement of blood clots in a patients body. Suitable Blood clot filters include, but are not limited to those described in U.S. Pat. Nos. 6,881,218; 6,623,507; 6,506,205; 6,497,709; 6,273,900; 6,214,025; 6,059,825, 6,007,558; 5,836,969; 5,836,968; 5,722,964; 5,669,933; 5,531,788; 5,413,586; 4,832,055; 4,817,600; 4,494,531; 4,425,908.

The blood clot filters may be coated with a coating selected from the group consisting of: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which don't have the capacity to synthesize their own asparagine); antiproliferative/antimitotic alkylating agents such as nitrogen mustards(mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (i.e. estrogen); Anticoagulants (heparin, synthetic heparin salts and other inhibitors of thrombin); fibrinolytic agents (such as tissue plasminogen activator, streptokinase and urokinase); antiplatelet: (aspirin, dipyridamole, ticlopidine, clopidogrel, abciximab); antimigratory; antisecretory (breveldin); antiinflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; Indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressive: (cyclosporine, tacrolimus (FK-506), sirolimus (rapamycin), azathioprine, mycophenolate mofetil); Angiogenic: any of the vascular endothelial growth factor(s) (VEGF), fibroblast growth factor (FGF); nitric oxide donors; anti-sense olgio nucleotides and combinations thereof.

Coating may be formulated by mixing one or more therapeutic agents with the coating polymers in a coating mixture. The therapeutic agent may be present as a liquid, a finely divided solid, or any other appropriate physical form. Optionally, the mixture may include one or more additives, e.g., nontoxic auxiliary substances such as diluents, carriers, excipients, stabilizers or the like.

Immediate release or controlled release coatings may be used. Immediate release coatings are those that will release substantially all pharmaceutically active agent in under an hour. Controlled release coatings are those that will release active agent over a period greater than an hour. For example, a controlled release coating may release substantially all active agent over a two, four, six, eight, twelve or twenty four hour period.

Any method of applying the coating to the blood clot filter known to one of ordinary skill in the art may be used. For example, methods of coating medical devices are disclosed for example, in U.S. Pat. Nos. 6,916,379; 6,860,946; 6,153,252; 6,106,473; 6,099,562; 5,922,393; 5,534,287.

All patents and patent applications referenced herein are specifically incorporated by reference in their entirety as though set forth in full.

I claim:

1. A blood clot filter for implantation in a patient comprising a blood clot filter having a coating comprising one or more pharmaceutically active compounds.

2. A blood clot filter according to claim 1 wherein the pharmaceutically active agent is selected from the group consisting of: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which don't have the capacity to synthesize their own asparagine); antiproliferative/antimitotic alkylating agents such as nitrogen mustards(mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin),trazenes-dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (i.e. estrogen); Anticoagulants (heparin, synthetic heparin salts and other inhibitors of thrombin); fibrinolytic agents (such as tissue plasminogen activator, streptokinase and urokinase); antiplatelet: (aspirin, dipyridamole, ticlopidine, clopidogrel, abciximab); antimigratory; antisecretory (breveldin); antiinflammatory: such as adrenocortical steroids (cortisol, cortisone, fludrocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives i.e. acetominophen; Indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressive: (cyclosporine, tacrolimus (FK-506), sirolimus (rapamycin), azathioprine, mycophenolate mofetil); Angiogenic: vascular endothelial growth factor (VEGF), fibroblast growth factor (FGF); nitric oxide donors; and, anti-sense olgio nucleotides.
